(19) United States
(12) Patent Application Publication
(10) Pub. No.: US 2020/0357162 A1
(43) Pub. Date: Nov. 12, 2020

(54) MODELING METHOD, APPARATUS, DEVICE AND STORAGE MEDIUM OF DYNAMIC CARDIOVASCULAR SYSTEM

(71) Applicant: BEIHANG UNIVERSITY, BEIJING (CN)

(72) Inventors: SHUAI LI, BEIJING (CN); AIMIN HAO, BEIJING (CN); QINPING ZHAO, BEIJING (CN)

(21) Appl. No.: 16/870,821

(22) Filed: May 8, 2020

(30) Foreign Application Priority Data
May 10, 2019 (CN) 201910390054.6

(51) Int. Cl. G06T 15/08; A61B 5/00; A61B 5/055; A61B 6/03; A61B 6/00; G06T 7/00

(52) U.S. Cl. CPC G06T 15/08; A61B 5/0044; A61B 5/055; G06T 2207/30048; A61B 6/504; A61B 6/5247; G06T 7/0012; A61B 6/032

(57) ABSTRACT

The present disclosure provides a modeling method, apparatus, device and storage medium of a dynamic cardiovascular system. The method includes: obtaining CMR data and CCTA data of a patient to be operated; constructing a dynamic ventricular model of the patient to be operated using the CMR data; constructing a dynamic heart model of the patient to be operated according to the dynamic ventricular model and a preset heart model; constructing a coronary artery model of the patient to be operated using the CCTA data; and constructing a dynamic cardiovascular system model of the patient to be operated according to the dynamic heart model and the coronary artery model, and constructing a personalized dynamic cardiovascular system model for different patients.

### CROSS-REFERENCE TO RELATED APPLICATIONS

The present application claims priority to Chinese patent application No. 201910390054.6, filed on May 10, 2019, which is incorporated by reference in its entirety.

### TECHNICAL FIELD

Embodiments of the present disclosure relate to the field of medical image processing technologies, and in particular, to a modeling method, apparatus, device and storage medium of a dynamic cardiovascular system.

### BACKGROUND

Cardiovascular diseases are one of leading causes of death worldwide. Among them, the coronary artery disease form plaques in coronary arteries, which is a serious threat to human life and health.

Percutaneous coronary intervention is an effective technique for treatment of the cardiovascular diseases, of which the main process is: under guidance of X-ray, a doctor directs a guide wire into the femoral artery by puncturing the blood vessel on the body surface. Through the femoral artery, the guide wire retrogradely enters into the aorta along the artery and enters the coronary vascular network. The doctor then diagnoses and treats symptoms such as coronary artery plunges with a specific cardiac catheterization technique. This is a minimally invasive surgery that is very complicated and generally requires excellent skills and rich experience for the surgeon.

In order to train a doctor's surgical skills and to perform rehearsal and planning for the surgical procedure, some specialized vascular intervention simulators have emerged. Dynamic virtual cardiovascular system models are required in the vascular intervention simulators. The existing dynamic virtual cardiovascular system models are all fixed models. Since there are differences in vascular structure and lesion location for different patients, use of a fixed dynamic virtual cardiovascular system model in a vascular intervention simulator can neither effectively improve the doctor's surgical skills nor accurately simulate avascular intervention required for each patient, thereby reducing authenticity of virtual training and rehearsal.

### SUMMARY

Embodiments of the present disclosure provide a modeling method, apparatus, device and storage medium of a dynamic cardiovascular system, which solve the technical problem in the prior that use of a fixed dynamic virtual cardiovascular system model in a vascular intervention simulator can neither effectively improve a doctor's surgical skills, nor accurately simulate a vascular intervention required for each patient, thereby reducing authenticity of virtual training and rehearsal.

In a first aspect, an embodiment of the present disclosure provides a modeling method of a dynamic cardiovascular system, including:

obtaining CMR data and coronary computed tomography angiography (CCTA) data of a patient to be operated;

constructing a dynamic ventricular model of the patient to be operatedusing the CMR data;

constructing a dynamic heart model of the patient to be operated according to the dynamic ventricular model and a preset heart model;

constructing a coronary artery model of the patient to be operated using the CCTA data; and

constructing a dynamic cardiovascular system model of the patient to be operated according to the dynamic heart model and the coronary artery model.

Further, in the method as described above, the constructing a dynamic ventricular model of the patient to be operated using the CMR data specifically includes:

constructing each frame of ventricular model using each frame of CMR data, where the ventricular model includes a plurality of vertices;

calculating a vertex correspondence between two adjacent frames of ventricular models using a registration algorithm; and

performing, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

Further, in the method as described above, the constructing a dynamic heart model of the patient to be operated according to the dynamic ventricular model and a preset heart model specifically includes:

constructing a filling ball model of the preset heart model;

establishing a mapping relationship between an element in the filling ball model and a surface vertex of the preset heart model;

determining a dynamic position of an element in the filling ball model according to the dynamic ventricular model;

determining a dynamic position of a surface vertex of the preset heart model according to the dynamic position of the element in the filling ball model; and

constructing a dynamic heart model of the patient to be operated according to the dynamic position of the surface vertex of the preset heart model.

Further, in the method as described above, the constructing a filling ball model of the preset heart model specifically includes:

triangulating the preset heart model to obtain a tetrahedral model;

providing a filling ballat avertex of the tetrahedral model, and connecting filling balls through a three-dimensional spring; and

determining a model composed of the filling ball and the three-dimensional spring as the filling ball model.

Further, in the method as described above, the constructing a coronary artery model of the patient to be operated using the CCTA data specifically includes:

performing coronary artery segmentation for the CCTA data using a level set algorithm to obtain a first coronary artery model;

extracting a center line in the first coronary artery model using a distance transform based three-dimensional center line extraction algorithm;

obtaining radius information of a cross section at each preset position of the center line; and

performing lofting processing on the center line according to the radius information of the cross section to obtain a second coronary artery model.

Further, in the method as described above, the constructing a dynamic cardiovascular system model of the patient to be operated according to the dynamic heart model and the coronary artery model specifically includes:

determining a cardiac portal for the coronary artery to enter in the dynamic heart model; and

registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.

Further, in the method as described above, after the registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated, further including:

correcting the dynamic cardiovascular system model of the patient to be operated using a shape matching algorithm.

In a second aspect, an embodiment of the present disclosure provides a modeling apparatus of a dynamic cardiovascular system, including:

a data obtaining module, configured to obtain CMR data and CCTA data of a patient to be operated;

a dynamic ventricular model constructing module, configured to construct a dynamic ventricular model of the patient to be operated using the CMR data;

a dynamic heart model constructing module, configured to construct a dynamic heart model of the patient to be operated according to the dynamic ventricular model and a preset heart model;

a coronary artery model constructing module, configured to construct a coronary artery model of the patient to be operated using the CCTA data; and

a dynamic cardiovascular system model constructing module, configured to construct a dynamic cardiovascular system model of the patient to be operated according to the dynamic heart model and the coronary artery model.

Further, in the apparatus as described above, the dynamic ventricular model constructing module is specifically configured to:

construct each frame of ventricular model using each frame of CMR data, where the ventricular model includes a plurality of vertices; calculate a vertex correspondence between two adjacent frames of ventricular models using a registration algorithm; and perform, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

Further, in the apparatus as described above, the dynamic heart model constructing module specifically includes:

a filling ball model constructing sub-module, configured to construct a filling ball model of the preset heart model;

a mapping relationship establishing sub-module, configured to establish a mapping relationship between an element in the filling ball model and a surface vertex of the preset heart model;

an element dynamic position determining sub-module, configured to determine a dynamic position of an element in the filling ball model according to the dynamic ventricular model;

a vertex dynamic position determining sub-module, configured to determine a dynamic position of a surface vertex of the preset heart model according to the dynamic position of the element in the filling ball model; and

a dynamic heart model constructing sub-module, configured to construct adynamic heart model of the patient to be operated according to the dynamic position of the surface vertex of the preset heart model.

Further, in the apparatus as described above, the filling ball model constructing sub-module is specifically configured to:

triangulate the preset heart model to obtain a tetrahedral model; provide a filling ball at a vertex of the tetrahedral model, and connect filling balls through a three-dimensional spring; and determine a model composed of the filling ball and the three-dimensional spring as the filling ball model.

Further, in the apparatus as described above, the coronary artery model constructing module is specifically configured to:

perform coronary artery segmentation for the CCTA data using a level set algorithm to obtain a first coronary artery model; extract a center line in the first coronary artery model using a distance transform based three-dimensional center line extraction algorithm; obtain radius information of a cross section at each preset position of the center line; and perform lofting processing on the center line according to the radius information of the cross section to obtain a second coronary artery model.

Further, in the apparatus as described above, the dynamic cardiovascular system model constructing module is specifically configured to:

determine a cardiac portal for the coronary artery to enter in the dynamic heart model; register the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.

Further, the apparatus as described above further includes:

a dynamic cardiovascular system model correcting module, configured to correct the dynamic cardiovascular system model of the patient to be operated using a shape matching algorithm.

In a third aspect, an embodiment of the present disclosure provides a terminal device, including:

a memory, a processor and a computer program;

where the computer program is stored in the memory and configured to be executable by the processor to implement the method according to any implementation of the first aspect above.

In a fourth aspect, an embodiment of the present disclosure provides a computer readable storage medium having a computer program stored thereon, where the computer program is executed by a processor to implement the method according to any implementation of the fourth aspect.

Embodiments of the present disclosure provide a modeling method, apparatus, device and storage medium of a dynamic cardiovascular system, which allow for obtaining CMR data and CCTA data of a patient to be operated; constructing a dynamic ventricular model of the patient to be operated using the CMR data; constructing a dynamic heart model of the patient to be operated according to the dynamic ventricular model and a preset heart model; constructing a coronary artery model of the patient to be operated using the CCTA data; and constructing a dynamic cardiovascular system model of the patient to be operated according to the dynamic heart model and the coronary artery model, therefore, a personalized dynamic cardiovascular system model can be constructed for a different patient, and arrangement of the personalized dynamic cardiovascular system model into a vascular intervention simulator can effectively improve a doctor's surgical skills and accurately simulate a vascular intervention required for each patient, thereby improving authenticity of virtual training and rehearsal.

It is to be understood that the content described above in the summary is neither intended to limit key or important feature sin embodiments of the present disclosure, nor to limit the scope of the present disclosure. Other features of the present disclosure will become readily comprehensible from the following description.

### BRIEF DESCRIPTION OF DRAWINGS

In order to more clearly illustrate technical solutions in the embodiments of the present disclosure or in the prior art, a brief introduction of accompanying drawings used for describing the embodiments or the prior art will be made below. Obviously, the accompanying drawings in the following description show some embodiments of the present disclosure, and those skilled in the art may still derive other drawings from these accompanying drawings without any creative effort.

FIG. 1 is a flowchart illustrating a modeling method of a dynamic cardiovascular system according to Embodiment 1 of the present disclosure;

FIG. 2 is a flowchart illustrating a modeling method of a dynamic cardiovascular system according to Embodiment 2 of the present disclosure;

FIG. 3 is a flowchart illustrating Step 202 in the modeling method of the dynamic cardiovascular system according to Embodiment 2 of the present disclosure;

FIG. 4 is a flowchart illustrating Step 203 in the modeling method of the dynamic cardiovascular system according to Embodiment 2 of the present disclosure;

FIG. 5 is a schematic diagram showing a mapping relationship between an element in a filling ball model and a surface vertex of a preset heart model according to Embodiment 2 of the present disclosure;

FIG. 6 is a flowchart illustrating Step 204 in the modeling method of the dynamic cardiovascular system according to Embodiment 2 of the present disclosure;

FIG. 7 is a schematic diagram showing a center line in the first coronary artery model extracted in Embodiment 2 of the present disclosure;

FIG. 8 is a schematic diagram showing a cross section at each preset position of the center line obtained in Embodiment 2 of the present disclosure;

FIG. 9 is a schematic diagram showing a second coronary artery model in Embodiment 2 of the present disclosure;

FIG. 10 is a flowchart illustrating Step 205 in a modeling method of a dynamic cardiovascular system according to Embodiment 2 of the present disclosure;

FIG. 11 is a schematic structural diagram of a modeling apparatus of a dynamic cardiovascular system according to Embodiment 3 of the present disclosure;

FIG. 12 is a schematic structural diagram of a modeling apparatus of a dynamic cardiovascular system according to Embodiment 4 of the present disclosure; and

FIG. 13 is a schematic structural diagram of a terminal device according to Embodiment 5 of the present disclosure.

### DESCRIPTION OF EMBODIMENTS

Embodiments of the present disclosure will be described in more detail below with reference to the accompanying drawings. Although some embodiments of the present disclosure are shown in the drawings, it should be understood that the present disclosure may be implemented in various forms, and should not be constructed as being limited to the embodiments illustrated herein. Instead, these embodiments are provided to fully and comprehensively understand the present disclosure. It will be appreciated that the drawings and the embodiments of the present disclosure are intended to be illustrative only but not to limit the scope of the present disclosure.

The terms such as “first”, “second”, “third”, “fourth” and the like (if any) in the specification and the claims of the present disclosure and in the drawings described above are used to distinguish similar objects rather to describe a specific order or sequence. It should be understood that data used in this way is interchangeable where appropriate so that the embodiments of the present disclosure described herein can be implemented in a sequence other than those illustrated or described herein. Moreover, terms such as “including” and “having” and any variations thereof are intended to cover a non-exclusive inclusion. For example, processes, methods, systems, products, or devices that include a series of steps or units are not necessarily limited to those steps or units that are clearly listed, but may include other steps or units that are not clearly listed or that are inherent to these processes, methods, products or devices.

In order to clearly understand the technical solutions of the present application, algorithms involved in the present application will be explained below:

CMR data: cardiovascular magnetic resonance data in full name. The CMR data is dynamic four-dimensional magnetic resonance data, and CMR data of each frame may be obtained.

CCTA data: coronary computed tomography angiography data in full name. The CCTA data is static three-dimensional data.

Embodiments of the present application will be specifically described below with reference to the drawings.

### Embodiment 1

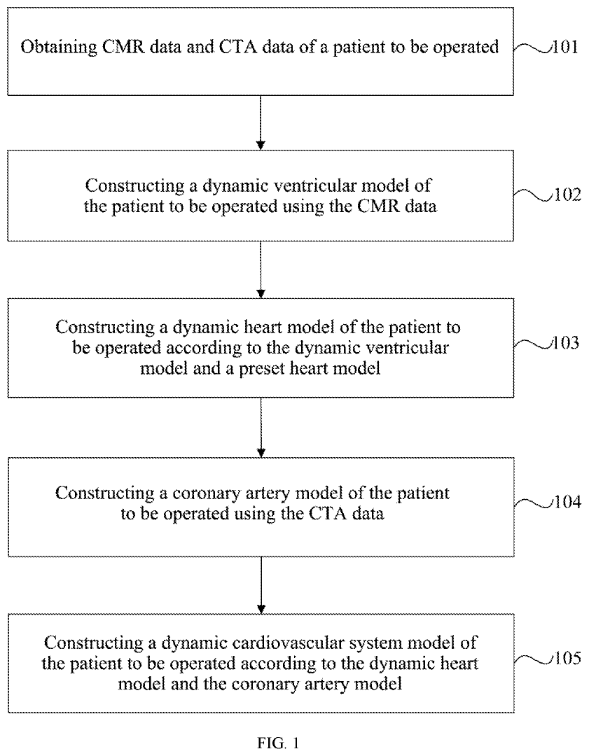

FIG. 1 is a flowchart illustrating a modeling method of a dynamic cardiovascular system according to Embodiment 1 of the present disclosure. As shown in FIG. 1, an execution body of the present embodiment is a modeling apparatus of a dynamic cardiovascular system, which may be integrated on a device with independent computing and processing capabilities, such as a computer, a laptop or a server. The modeling method of the dynamic cardiovascular system provided in the present embodiment includes the following steps.

Step 101, obtaining CMR data and CCTA data of a patient to be operated.

Specifically, in the present embodiment, a patient to be operated is performed with nuclear magnetic resonance scanning to form CMR data so that the CMR data is obtained;

and the patient to be operated is performed with coronary computerized tomography scanning to form CCTA data so that the CCTA data is obtained.

The CMR data is four-dimensional magnetic resonance data including multiple frames of magnetic resonance image sequence data. The CCTA data is three-dimensional image data.

Step 102, constructing a dynamic ventricular model of the patient to be operated using the CMR data.

Specifically, in the present embodiment, each frame of magnetic resonance image undergoing CMR is first divided to obtain ventricular magnetic resonance images, and then each frame of ventricular model is constructed according to each frame of ventricular magnetic resonance image. Respective frames of ventricular models form a discrete ventricular model, and finally the discrete respective frames of ventricular models are allowed to be continuous to form a dynamic ventricular model.

In the present embodiment, the method based on which each frame of ventricular model is constructed according to each frame of ventricular magnetic resonance image may include: firstly, constructing a compact and relatively accurate anatomical prior model according to information such as the position, size, and shape in the ventricle, where the anatomical prior model is formed by connecting a plurality of tetrahedrons, each of which has a plurality of vertices so that the anatomical prior model includes a plurality of vertices; and then using a registration algorithm to calculate a deformation field from the anatomical prior model to each frame of ventricular magnetic resonance image so that each frame of ventricular model is obtained. Therefore, each frame of ventricular model also includes a plurality of vertices.

In the present embodiment, for the method based on which each frame of ventricular model is constructed according to each frame of ventricular magnetic resonance image, there may be other methods, which will not be limited in the present embodiment.

Step 103, constructing a dynamic heart model of the patient to be operated according to the dynamic ventricular model and a preset heart model.

The preset heart model may be a heart model constructed subsequent to statistical analysis of a large number of hearts in terms of their shapes, size and positions. The preset heart model is a model based on a triangular patch.

Specifically, in the present embodiment, the method based on which a dynamic heart model of the patient to be operated is constructed according to the dynamic ventricular model and a preset heart model may include: firstly, establishing a mapping relationship between each vertex of each frame of ventricular model and a surface vertex of the preset heart model; adjusting the position of the surface vertex of the preset heart model according to the mapping relationship between each vertex of each frame of ventricular model and the surface vertex of the preset heart model as well as the position of each vertex in each frame of ventricular model to obtain each frame of heart model; and allowing respective frames of heart models to be continuous to form a dynamic heart model of the patient to be operated.

Specifically, in the present embodiment, the method based on which a dynamic heart model of the patient to be operated is constructed according to the dynamic ventricular model and a preset heart model may include: constructing a filling ball model of the preset heart model; establishing a mapping relationship between an element in the filling ball model and a surface vertex in the preset heart model; determining a dynamic position of an element in the filling ball model according to the dynamic ventricular model; determining a dynamic position of a surface vertex in the preset heart model according to the dynamic position of the element in the filling ball model; and constructing a dynamic heart model of the patient to be operated according to the dynamic position of the surface vertex in the preset heart model.

In the present embodiment, for the method based on which a dynamic heart model of the patient to be operated is constructed according to the dynamic ventricular model and a preset heart model, there may be other methods, which will not be limited in the present embodiment.

Step 104, constructing a coronary artery model of the patient to be operated using the CCTA data.

Specifically, in the present embodiment, the method based on which a coronary artery model of the patient to be operated is constructed using the CCTA data may include: segmenting the coronary artery in the CCTA data using a segmentation algorithm, and then refining the segmented coronary artery to obtain a fine coronary artery.

The segmentation algorithm may be a level set algorithm or other segmentation algorithm, which will not be limited in the present embodiment. The method based on which the segmented coronary artery is refined may include: extracting a vascular center line; obtaining radius information of a cross section at a preset distance from the center line; and performing lofting processing on the center line according to the radius information of the cross section to obtain a refined fine coronary artery.

Step 105, constructing a dynamic cardiovascular system model of the patient to be operated according to the dynamic heart model and the coronary artery model.

Specifically, in the present embodiment, the coronary artery model is registered into the dynamic heart model, such that the coronary artery model enters the dynamic heart model along the cardiac portal and is attached to the surface of the dynamic heart model, and left and right vessel branches correspond to corresponding ventricles of the dynamic heart model, resulting in a dynamic cardiovascular system model of the patient to be operated.

The modeling method of the dynamic cardiovascular system model provided in the present embodiment allows for obtaining CMR data and CCTA data of a patient to be operated; constructing a dynamic ventricular model of the patient to be operated using the CMR data; constructing a dynamic heart model of the patient to be operated according to the dynamic ventricular model and a preset heart model; constructing a coronary artery model of the patient to be operated using the CCTA data; and constructing a dynamic cardiovascular system model of the patient to be operated according to the dynamic heart model and the coronary artery model, therefore, a personalized dynamic cardiovascular system model can be constructed for a different patient, and arrangement of the personalized dynamic cardiovascular system model into a vascular intervention simulator can effectively improve a doctor's surgical skills and accurately simulate a vascular intervention required for each patient, thereby improving authenticity of virtual training and rehearsal.

### Embodiment 2

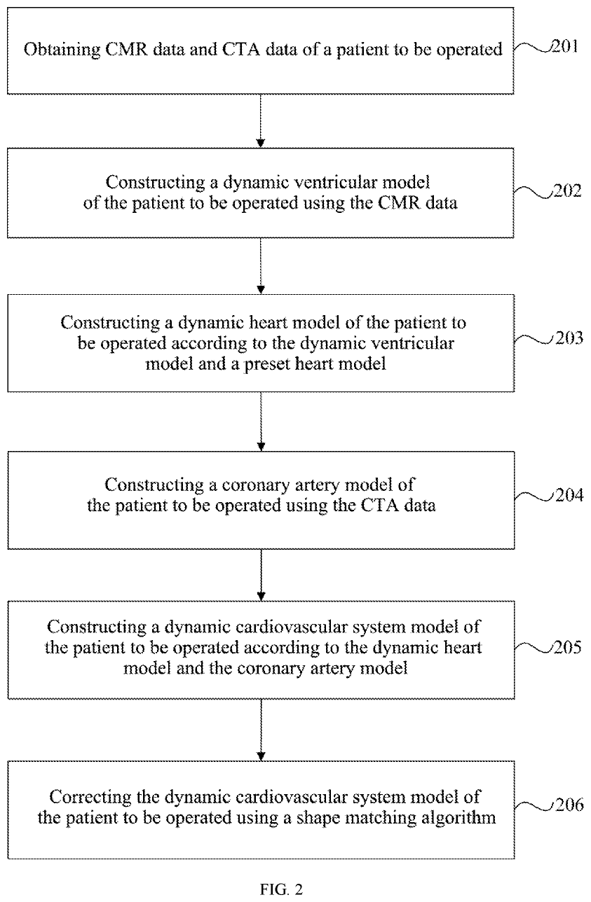

FIG. 2 is a flowchart illustrating a modeling method of a dynamic cardiovascular system according to Embodiment 2 of the present disclosure. As shown in FIG. 2, the modeling method of the dynamic cardiovascular system provided in the present embodiment is intended to further refine Step 102 to Step 105 based on the modeling method of the dynamic cardiovascular system provided in Embodiment 1 of the present disclosure, and then the modeling method of the dynamic cardiovascular system provided in the present embodiment includes the following steps.

Step 201, obtaining CMR data and CCTA data of a patient to be operated.

In the present embodiment, the implementation of Step 201 is the same as that of Step 101 in the modeling method of the dynamic cardiovascular system provided in Embodiment 1 of the present disclosure, which will not be repeated herein.

Step 202, constructing a dynamic ventricular model of the patient to be operated using the CMR data.

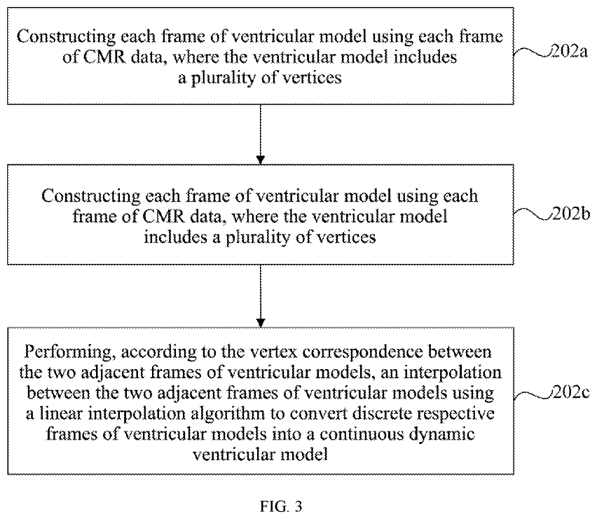

Further, in the present embodiment, FIG. 3 is a flowchart illustrating Step 202 in the modeling method of the dynamic cardiovascular system according to Embodiment 2 of the present disclosure. As shown in FIG. 3, in the present embodiment, Step 202 in which a dynamic ventricular model of the patient to be operated is constructed using the CMR data specifically includes three steps as follows.

Step 202a, constructing each frame of ventricular model using each frame of CMR data, where the ventricular model includes a plurality of vertices.

Further, in the present embodiment, an anatomical prior model is first constructed, and then morphological variability in the anatomical prior model is parameterized; let x={xⱼ; i=1, . . . , n} be n marked bodies, and each body is described by m three-dimensional marker points in series and expressed as pⱼ=[p₁ⱼ, p₂ⱼ, p₃ⱼ] (j=1, . . . , m). Then x is distributed in the 3m dimensional space. The marked body model can be expressed as Formula (1).

x=x̅+φb (1)

Where

$$\overline{x} = \frac{1}{n} {\sum }_{i = 1}^{n} x$$

is an average marker point vector; b is a body model parameter vector; φ is a matrix composed of eigenvectors corresponding to the covariance matrix

$$S = \frac{1}{n - 1} {\sum }_{i = 1}^{n} ( {x}_{i} - \overline{x} ) {( {x}_{i} - \overline{x} )}^{T} .$$

Eigen values λᵢ of S are ordered, such that λᵢ≥λᵢ₊₁, φ is a matrix composed of eigenvectors corresponding to t largest non-zero eigen values of S, where t=min{m, n}.

In the present embodiment, Formula (1) can be used to represent each frame of ventricular model X , here φ=[φ₁, φ₂, . . . , φₜ], b is a t-dimensional vector, and b=φ^{T}(x−x̅).

Step 202b, calculating a vertex correspondence between two adjacent frames of ventricular models using a registration algorithm.

Step 202c, performing, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

Further, in the present embodiment, after each frame of ventricular model is obtained, calculate a vertex correspondence between two adjacent frames of ventricular models using a registration algorithm, and perform, according to the vertex correspondence between the two adjacent frames of ventricular models, an interpolation between corresponding vertices in the two adjacent frames of ventricular models to convert discrete respective frames of ventricular models into a continuous dynamic ventricular model.

Where the interpolation algorithm is a linear interpolation algorithm.

Step 203, constructing a dynamic heart model of the patient to be operated according to the dynamic ventricular model and a preset heart model.

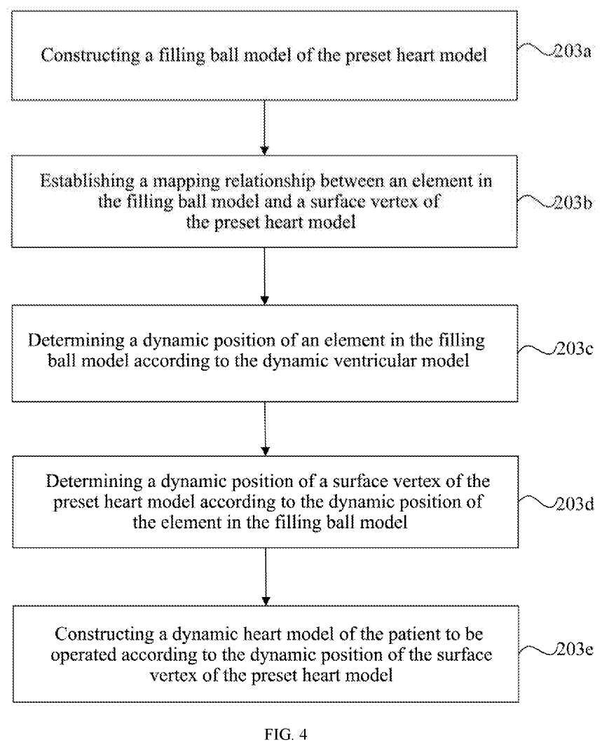

Further, in the present embodiment, FIG. 4 is a flowchart illustrating Step 203 in the modeling method of the dynamic cardiovascular system according to Embodiment 2 of the present disclosure. As shown in FIG. 4, in the present embodiment, Step 203 in which a dynamic heart model of the patient to be operated is constructed according to the dynamic ventricular model and a preset heart model specifically includes three steps as follows.

Step 203a, constructing a filling ball model of the preset heart model.

Further, in the present embodiment, the constructing a filling ball model of the preset heart model specifically includes:

Firstly, triangulating the preset heart model to obtain a tetrahedral model;

Secondly, providing a filling ballat a vertex of the tetrahedral model, and connecting filling balls through a three-dimensional spring;

Finally, determining a model composed of the filling ball and the three-dimensional spring as the filling ball model.

Specifically, in the present embodiment, the preset heart model is based on a triangular patch, and it needs to be triangulated to obtain a volume attribute, where the triangulation may be Delaunary triangulation, so as to obtain a tetrahedral model. Elastic filling balls are then placed at vertices of each tetrahedron of each tetrahedral model, and these filling balls are connected through a three-dimensional spring that is resistant to bending, tension, and torsion. Finally, a model composed of the filling ball and the three-dimensional spring is determined as the filling ball model.

In the present embodiment, the constructing the filling ball model of the preset heart model can decouple local deformation and global deformation, and collision detection may be performed efficiently, which is suitable for real-time application.

Step 203b, establishing a mapping relationship between an element in the filling ball model and a surface vertex of the preset heart model.

Specifically, in the present embodiment, the element in the filling ball model is a three-dimensional spring connecting the filling balls. In the present embodiment, a mapping relationship between an element in the filling ball model and a surface vertex of the preset heart model is established according to a spatial positional relationship of points in Euclidean space.

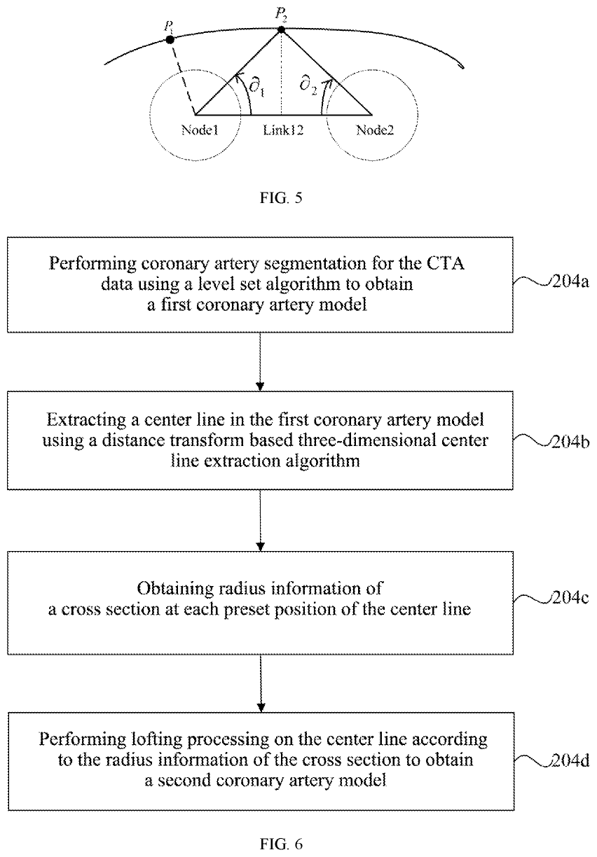

FIG. 5 is a schematic diagram showing the mapping relationship between the element in the filled ball model and the surface vertex of the preset heart model according to Embodiment 2 of the present disclosure. As shown in FIG. 5, the filling balls are respectively Node1 and Node2, and the three-dimensional spring connecting the two filling balls is Link12. Surface vertices of the preset heart model are P₁, P₂. A three-dimensional spring of a filling ball closest to the surface vertices P₁, P₂ or a three-dimensional spring between the filling balls is determined according to a spatial positional relationship of points in Euclidean space. Where a mapping relationship with Node1 is established for point P₁, angles between P₂ and Link12 connecting the two filling balls are ∂₁<90° and ∂₂<90°, since P₂ is closest to Link12 in Euclidean distance, a mapping relationship with Link12 is established for point P₂.

In the present embodiment, the establishing a mapping relationship between an element in the filling ball model and a surface vertex of the preset heart model may also achieve some functions in surgical simulation, including physical movements of human organs and medical instruments, collision detection and collision response between organs and devices, high-precision drawing and rendering of organs and instruments.

Step 203c, determining a dynamic position of an element in the filling ball model according to the dynamic ventricular model.

Specifically, in the present embodiment, association mapping between the dynamic ventricular model and the filling ball model is established using a mapping method. Specifically, although the dynamic ventricular model and the filling ball model have different topological structures, their overall shape and size are basically similar, that is, there is similarity in spatial position, therefore, the mapping is established through the spatial positional relationship of the points in Euclidean space. In a mapping relationship constructing phase, for each Node and Link in the filling ball model, the closest point on the dynamic ventricular model is searched within a certain domain as an association point, so as to complete the mapping association between the two models. When a certain vertex of the dynamic ventricular model changes in spatial position, the associated filling ball model changes accordingly.

In the present embodiment, the dynamic ventricular model is used as the driving force to drive the position of the element in the filling ball model to be changed. The filling ball model decouples local deformation and global deformation, and the driving force of the dynamic ventricular model first causes a local deformation for the filling ball model and then gradually causes a deformation for the global model. The position of each element in the filling ball model is determined when the position of the surface vertex of the dynamic ventricular model changes at each frame.

Step 203d, determining a dynamic position of a surface vertex of the preset heart model according to the dynamic position of the element in the filling ball model.

Further, let the filling ball Node1 have a position coordinate of n₁ at the center thereof, and a rotation matrix of rot₁ around the center thereof Node2 has a position coordinate of n₂ at the center thereof, and a rotation matrix of rot₂ around the center thereof. For a local coordinate system whose coordinate center is at position n₁, the rotation matrix is determined according to Formulas (2), (3) and (4):

$$\begin{matrix}{A}_{0} = {rot}_{1}^{T} \cdot \frac{( {n}_{2} - {n}_{1} ) \times ( 0 , 1 , 0 )}{\vert ( {n}_{2} - {n}_{1} ) \times ( 0 , 1 , 0 ) \vert} & ( 2 ) \\ {B}_{0} = r o {t}_{1}^{T} \cdot \frac{( {n}_{2} - {n}_{1} ) \times {A}_{0}}{( {n}_{2} - {n}_{1} ) \times {A}_{0}} & ( 3 ) \\ {C}_{0} = ( {n}_{2} - {n}_{1} ) & ( 4 )\end{matrix}$$

Where Formula (2) is the X-axis coordinate, Formula (3) is the Y-axis coordinate, and Formula (4) is the Z-axis coordinate. Suppose that one point on the surface of the preset heart model has a position coordinate of pos . If this point has a mapping relationship with Node1, its position is updated by rot₁^{T}·(pos-n₁); if this point has a mapping relationship with Link12 , its position is updated by (A₀, B₀, C₀)⁻¹·(pos-n₁) . Where (A₀, B₀, C₀) is a rotation matrix constructed by taking A₀, B₀, C₀ as column vectors. In a dynamics simulation process, as long as rotation matrixes of all filling balls are updated in real time, surface vertices of the preset heart model having a mapping relationship with the rotation matrixes can be driven to be changed in positions, thereby determining a dynamic position of a surface vertex of the preset heart model according to the dynamic position of the element in the filling ball model.

Step 203e, constructing a dynamic heart model of the patient to be operated according to the dynamic position of the surface vertex of the preset heart model.

Further, a heart model at each moment is constructed according to the position of the surface vertex of the preset heart model at that moment, and heart models at respective moments are allowed to be continuous to form a dynamic heart model of the patient to be operated.

Step 204, constructing a coronary artery model of the patient to be operated using the CCTA data.

Further, FIG. 6 is a flowchart illustrating Step 204 in the modeling method of the dynamic cardiovascular system according to Embodiment 2 of the present disclosure. As shown in FIG. 6, in the present embodiment, Step 204 in which a coronary artery model of the patient to be operated is constructed using the CCTA data includes four steps as follows.

Step 204a, performing coronary artery segmentation for the CCTA data using a level set algorithm to obtain a first coronary artery model.

Further, since the level set algorithm can simultaneously describe topology and shape changes, boundaries remain smooth during an optimization process. Therefore, in the present embodiment, the level set algorithm is used to perform coronary artery segmentation for a three-dimensional image undergoing CCTA. The basic idea is to embed the evolution curve or surface, as a zero-level set, into the higher one-dimensional level set function, and obtain the evolution equation of the level set function by solving the surface evolution equation.

Specifically, a seed point is set first in a region of interest to initialize the level set, and the segmented result is obtained by solving the partial differential equation and then obtaining a unique solution that satisfies the condition. Where a GPU-based parallel method is used for solving the partial differential equation. Finally, a first coronary artery model may be obtained by cropping the segmented result.

Where the partial differential equation is expressed as Formula (5).

$$\{ \begin{matrix}\frac{\partial φ}{\partial t} = F ( K ) \vert ∇ φ \vert \\ φ ( {C}_{0} ( p ) , 0 ) = \pm d\end{matrix} \qquad ( 5 )$$

Where C₀(p) is an initial two-dimensional closed curve, and it is a closed curve moving and evolving along its inner normal vector N at time t, ϕ is a symbol distance function; F(K)is a velocity function; K is curvature; and d is the distance from point p to the curve, whether it is positive or negative depends on the fact that p is located on the outside or on the inside of the curve. Due to introduction of the symbol distance function and the velocity function, the evolution of the high-dimensional function not only utilizes global information of the image, but also utilizes local information of the image. Therefore, it has great advantages in dealing with a target whose topological shape changes.

In the present embodiment, the coronary artery segmentation for the CCTA data using the level set algorithm is a rough segmentation, and the first coronary artery model obtained accordingly is a rough coronary artery model.

Step 204b, extracting a center line in the first coronary artery model using a distance transform based three-dimensional centerline extraction algorithm.

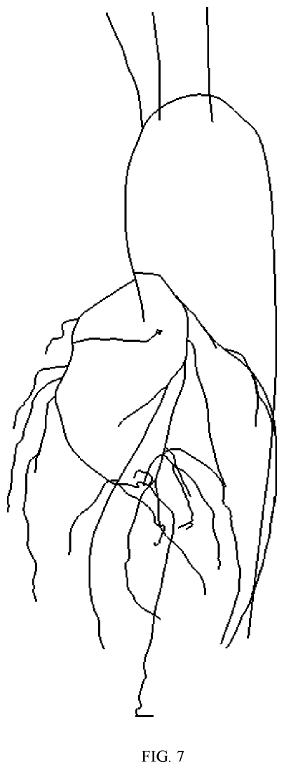

Further, since the structure in the first coronary artery model is relatively rough and is not suitable for direct surgery simulation, refinement processing is performed on the basis of the first coronary artery model. FIG. 7 is a schematic diagram showing a center line in the first coronary artery model extracted in Embodiment 2 of the present disclosure. As shown in FIG. 7, when a center line in the first coronary artery model is extracted using a distance transform based three-dimensional center line extraction algorithm, the first coronary artery model is firstly scanned twice in different directions for distance transformation, the first coronary artery model is then scanned once in a different direction for extraction of points on the center line, and the points on the center line are connected to form the centerline in the first coronary artery model.

Step 204c, obtaining radius information of a cross section at each preset position of the center line.

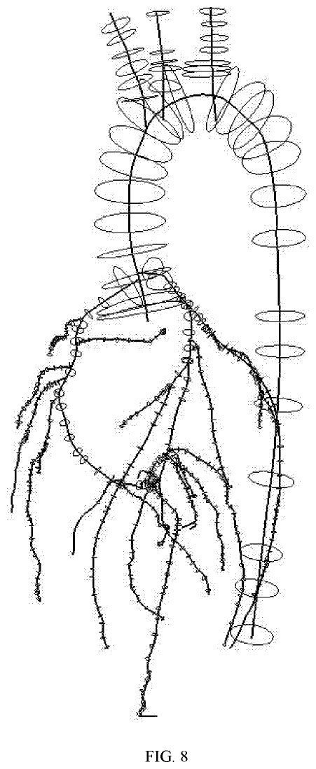

Further, in the present embodiment, FIG. 8 is a schematic diagram showing a cross section at each preset position of the center line obtained in Embodiment 2 of the present disclosure. As shown in FIG. 8, a position for obtaining a cross section is determined along the center line at each preset distance, and radius information of the cross section at each position is obtained according to edge characteristics of blood vessels in the three-dimensional image undergoing CCTA.

Step 204d, performing lofting processing on the center line according to the radius information of the cross section to obtain a second coronary artery model.

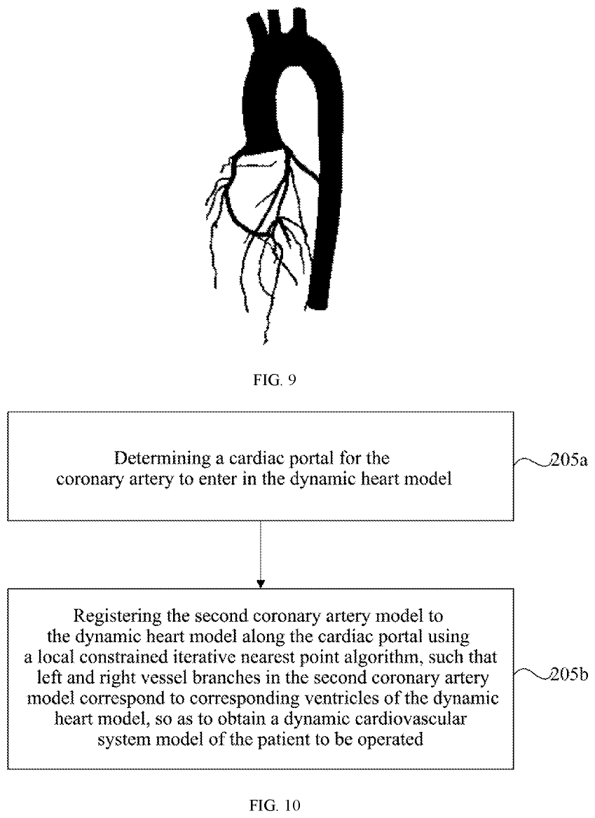

Further, FIG. 9 is a schematic diagram showing a second coronary artery model in Embodiment 2 of the present disclosure. As shown in FIG. 9, in the present embodiment, the second coronary artery model is a more refined coronary artery model than the first coronary artery model.

Step 205, constructing a dynamic cardiovascular system model of the patient to be operated according to the dynamic heart model and the coronary artery model.

FIG. 10 is a flowchart illustrating Step 205 in a modeling method of a dynamic cardiovascular system according to Embodiment 2 of the present disclosure. As shown in FIG. 10, further, in the present embodiment, Step 205 in which a dynamic cardiovascular system model of the patient to be operated is constructed according to the dynamic heart model and the coronary artery model includes the following steps.

Step 205a, determining a cardiac portal for the coronary artery to enter in the dynamic heart model.

Further, in the present embodiment, the dynamic heart model and the second coronary artery model are combined and registered to form a dynamic cardiovascular system model. To simplify the registration process, a segment of geometric model is assigned in the dynamic heart model as a cardiac portal for coronary artery to enter.

Step 205b, registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.

Further, in the present embodiment, a local constrained iterative nearest point algorithm is used to register the second coronary artery model to the dynamic heart model along the cardiac portal, simultaneously, constraints are applied to enable left and right vessel branches in the second coronary artery model to correspond to corresponding ventricles of the dynamic heart model, resulting in a dynamic cardiovascular system model that can be used for simulation of a percutaneous coronary intervention.

Step 206, correcting the dynamic cardiovascular system model of the patient to be operated using a shape matching algorithm.

Further, in the present embodiment, since when a filling ball model is used, the force first acts on a filling ball node, a new position of the filling ball is obtained by Euler's second order integral; at this time, the link connecting filling balls changes in relation to its initial state; all links are traversed to obtain the corresponding bending resistant moment, torsion resistant moment and tensile resistance; these forces are then applied to filling ball nodes connected by the link. The deformation is transferred from local to global based on such steps, resulting in hysteresis of the global deformation. Therefore, a shape matching algorithm is used in the present embodiment to correct the dynamic cardiovascular system model of the patient to be operated, avoiding the hysteresis of the global deformation of the dynamic cardiovascular system model and improving the global deformation effect of the dynamic cardiovascular model.

### Embodiment 3

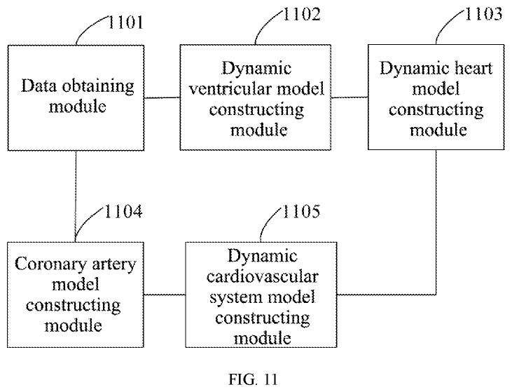

FIG. 11 is a schematic structural diagram of a modeling apparatus of a dynamic cardiovascular system model according to Embodiment 3 of the present disclosure. As shown in FIG. 11, the modeling apparatus of the dynamic cardiovascular system provided in the present embodiment includes: a data obtaining module 1101, a dynamic ventricular model constructing module 1102, a dynamic heart model constructing module 1103, a coronary artery model constructing module 1104 and a dynamic cardiovascular system model constructing module 1105.

Where the data obtaining module 1101 is configured to obtain CMR data and CCTA data of a patient to be operated. The dynamic ventricular model constructing module 1102 is configured to construct a dynamic ventricular model of the patient to be operated using the CMR data. The dynamic heart model constructing module 1103 is configured to construct a dynamic heart model of the patient to be operated according to the dynamic ventricular model and a preset heart model. The coronary artery model constructing module 1104 is configured to construct a coronary artery model of the patient to be operated using the CCTA data. The dynamic cardiovascular system model constructing module 1105 is configured to construct a dynamic cardiovascular system model of the patient to be operated according to the dynamic heart model and the coronary artery model.

The modeling apparatus of the dynamic cardiovascular system provided in the present embodiment may perform the technical solution of the method embodiment shown in FIG. 1, and implementation principles and technical effects thereof are similar to those for the method embodiment, which will not be repeated herein.

### Embodiment 4

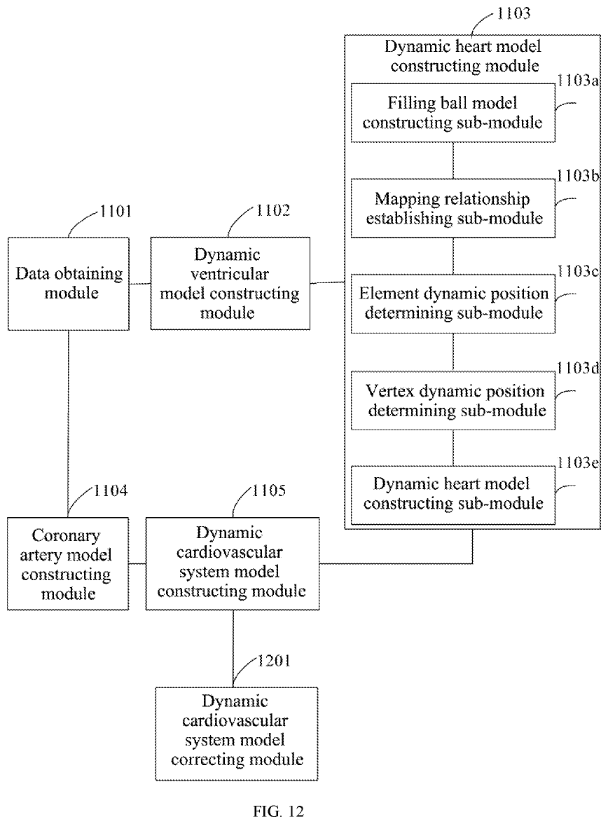

FIG. 12 is a schematic structural diagram of a modeling apparatus of a dynamic cardiovascular system according to Embodiment 4 of the present disclosure. As shown in FIG. 12, based on the modeling apparatus of the dynamic cardiovascular system provided in Embodiment 3 of the present disclosure, the modeling apparatus of the dynamic cardiovascular system provided in the present embodiment further includes a dynamic cardiovascular system model correcting module 1201.

Further, the dynamic ventricular model constructing module 1102 is specifically configured to: construct each frame of ventricular model using each frame of CMR data, where the ventricular model includes a plurality of vertices; calculate a vertex correspondence between two adjacent frames of ventricular models using a registration algorithm; and perform, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

Further, the dynamic heart model constructing module 1103 specifically includes: a filling ball model constructing sub-module 1103a, a mapping relationship establishing sub-module 1103b, an element dynamic position determining sub-module 1103c, a vertex dynamic position determining sub-module 1103d, and a dynamic heart model constructing sub-module 1103e.

Where the filling ball model constructing sub-module 1103a is configured to construct a filling ball model of the preset heart model. The mapping relationship establishing sub-module 1103b is configured to establish a mapping relationship between an element in the filling ball model and a surface vertex of the preset heart model. The element dynamic position determining sub-module 1103c is configured to determine a dynamic position of an element in the filling ball model according to the dynamic ventricular model. The vertex dynamic position determining sub-module 1103d is configured to determine a dynamic position of a surface vertex of the preset heart model according to the dynamic position of the element in the filling ball model. The dynamic heart model constructing sub-module 1103e is configured to construct a dynamic heart model of the patient to be operated according to the dynamic position of the surface vertex of the preset heart model.

Further, the filling ball model constructing sub-module 1103a is specifically configured to: triangulate the preset heart model to obtain a tetrahedral model; provide a filling ball at a vertex of the tetrahedral model, and connect filling balls through a three-dimensional spring; and determine a model composed of the filling ball and the three-dimensional spring as the filling ball model.

Further, the coronary artery model constructing module 1104 is specifically configured to: perform coronary artery segmentation for the CCTA data using a level set algorithm to obtain a first coronary artery model; extract a center line in the first coronary artery model using a distance transform based three-dimensional center line extraction algorithm; obtain radius information of a cross section at each preset position of the center line; and perform lofting processing on the center line according to the radius information of the cross section to obtain a second coronary artery model.

Further, the dynamic cardiovascular system model constructing module 1105 is specifically configured to: determine a cardiac portal for the coronary artery to enter in the dynamic heart model; register the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.

Further, the dynamic cardiovascular system model correcting module 1201 is configured to correct the dynamic cardiovascular system model of the patient to be operated using a shape matching algorithm.

The modeling apparatus of the dynamic cardiovascular system provided in the present embodiment may perform the technical solution of the method embodiment shown in FIG. 2, and implementation principles and technical effects thereof are similar to those for the method embodiment, which will not be repeated herein.

### Embodiment 5

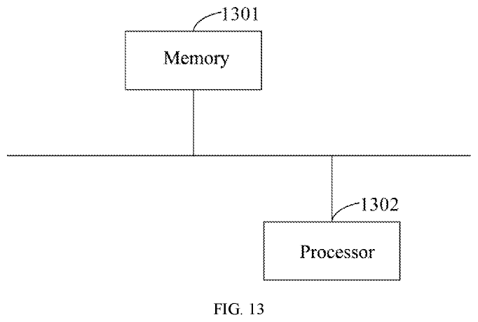

FIG. 13 is a schematic structural diagram of a terminal device according to Embodiment 5 of the present disclosure. As shown in FIG. 13, the terminal device according to an embodiment of the present disclosure includes: a memory 1301, a processor 1302 and a computer program.

Where the computer program is stored in the memory 1301 and is configured to be executable by the processor 1302 to implement the modeling method of the dynamic cardiovascular system provided in Embodiment 1 of the present disclosure or the modeling method of the dynamic cardiovascular system provided in Embodiment 2 of the present disclosure. Related illustrations can be understood by referring to relevant descriptions and effects corresponding to steps in FIG. 1 to FIG. 2, which will not be repeated herein.

### Embodiment 6

An embodiment of the present disclosure further provides a computer readable storage medium having a computer program stored thereon, where the computer program is executed by a processor to implement the modeling method of the dynamic cardiovascular system provided in Embodiment 1 of the present disclosure or provided in Embodiment 2 of the present disclosure.

In several embodiments provided in the present disclosure, it should be understood that the disclosed apparatus and method may be implemented in other manners. For example, the apparatus embodiments described above are merely illustrative. For example, the division of the module is only a logical function division, and there may be another division manner in actual implementation; for example, multiple modules or components may be combined or may be integrated into another system, or some features can be ignored or not be executed. In addition, the mutual coupling or direct coupling or communication connection shown or discussed may be implemented through some interfaces. The indirect coupling or communication connection between apparatuses or modules may be implemented in an electrical form, mechanical form or in other forms.

The units described as separate components may or may not be physically separated, and the components presented as modules may or may not be physical modules, that is, may be located in one place, or may be distributed to multiple network modules. Some or all the modules may be selected as required in order to achieve the purpose of the solution of the present embodiment.

In addition, functional modules in embodiments of the present disclosure may be integrated into one processing module, or each of the modules may exist alone separately, or two or more modules may be integrated into one module. The above integrated module can be implemented in the form of hardware or in the form of hardware plus software functional modules.

The program codes for implementing the method of the present disclosure may be written in any combination of one or more programming languages. Such program codes may be provided to a processor or a controller of a general purpose computer, a dedicated computer or other programmable data processing apparatus, such that the program codes, when executed by the processor or the controller, cause the functions/operations specified in the flowcharts and/or block diagrams to be implemented. The program codes may be executed entirely or partly on a machine, or may be executed, as a stand-alone software package, partly on a machine and partly on a remote machine, or executed entirely on a remote machine or a server.

In the context of the present disclosure, a machine-readable medium may be a tangible medium that may contain or store a program for use by or in combination with an instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination of the foregoing. More detailed examples of the machine readable storage media may include an electrically connected portable computer disk based on one or more wires, a hard disk, a random access memory (RAM), a read only memory (ROM), an erasable programmable read only memory (EPROM or a flash memory), an optical fiber, a portable compact disk read only memory (CD-ROM), an optical storage device, a magnetic storage device, or any suitable combination of the foregoing.

In addition, although the operations are depicted in a particular order, this should be understood that such operations are required to be performed in the particular order shown or in sequence, or that all illustrated operations should be performed to achieve desired results. Multitasking and parallel processing may be advantageous in certain circumstances. Likewise, although several specific implementation details are included in the above discussion, these should not be construed as limiting the scope of the disclosure. Certain features that are described in context of an individual embodiment may be implemented in combination in a single implementation. Conversely, various features that are described in context of a single implementation can be implemented in a plurality of implementations, either individually or in any suitable sub-combination.

Although the subject has been described in a language specific to structural features and/or methodological acts, it should be understood that the subject defined in the appended claims is not necessarily limited to the specific features or acts described above. Instead, the specific features and acts described above are merely exemplary forms for implementing the claims.

What is claimed is:

1. A modeling method of a dynamic cardiovascular system, comprising:
obtaining cardiovascular magnetic resonance (CMR) data and coronary computed tomography angiography(CCTA) data of a patient to be operated;
constructing a dynamic ventricular model of the patient to be operated using the CMR data;
constructing a dynamic heart model of the patient to be operated according to the dynamic ventricular model and a preset heart model;
constructing a coronary artery model of the patient to be operated using the CCTA data; and
constructing a dynamic cardiovascular system model of the patient to be operated according to the dynamic heart model and the coronary artery model.

2. The method according to claim 1, wherein the constructing a dynamic ventricular model of the patient to be operated using the CMR data comprises:
constructing each frame of ventricular model using each frame of CMR data, wherein the ventricular model comprises a plurality of vertices;
calculating a vertex correspondence between two adjacent frames of ventricular models using a registration algorithm; and
performing, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

3. The method according to claim 1, wherein the constructing a dynamic heart model of the patient to be operated according to the dynamic ventricular model and a preset heart model comprises:
constructing a filling ball model of the preset heart model;
establishing a mapping relationship between an element in the filling ball model and a surface vertex of the preset heart model;
determining a dynamic position of an element in the filling ball model according to the dynamic ventricular model;
determining a dynamic position of a surface vertex of the preset heart model according to the dynamic position of the element in the filling ball model; and
constructing a dynamic heart model of the patient to be operated according to the dynamic position of the surface vertex of the preset heart model.

4. The method according to claim 3, wherein the constructing a filling ball model of the preset heart model comprises:
triangulating the preset heart model to obtain a tetrahedral model;
providing a filling ballat a vertex of the tetrahedral model, and connecting filling balls through a three-dimensional spring; and
determining a model composed of the filling ball and the three-dimensional spring as the filling ball model.

5. The method according to claim 1, wherein the constructing a coronary artery model of the patient to be operated using the CCTA data comprises:
performing coronary artery segmentation for the CCTA data using a level set algorithm to obtain a first coronary artery model;
extracting a center line in the first coronary artery model using a distance transform based three-dimensional center line extraction algorithm;
obtaining radius information of a cross section at each preset position of the center line; and
performing lofting processing on the center line according to the radius information of the cross section to obtain a second coronary artery model.

6. The method according to claim 5, wherein the constructing a dynamic cardiovascular system model of the patient to be operated according to the dynamic heart model and the coronary artery model comprises:
determining a cardiac portal for the coronary artery to enter in the dynamic heart model; and
registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.

7. The method according to claim 6, wherein after the registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated, further comprising:
correcting the dynamic cardiovascular system model of the patient to be operated using a shape matching algorithm.

8. A terminal device, comprising:
a memory, a processor and a computer program;
wherein the computer program is stored in the memory and configured to be executable by the processor to implement the following steps:
obtaining cardiovascular magnetic resonance (CMR) data and coronary computed tomography angiography(CCTA) data of a patient to be operated;
constructing a dynamic ventricular model of the patient to be operated using the CMR data;
constructing a dynamic heart model of the patient to be operated according to the dynamic ventricular model and a preset heart model;
constructing a coronary artery model of the patient to be operated using the CCTA data; and
constructing a dynamic cardiovascular system model of the patient to be operated according to the dynamic heart model and the coronary artery model.

9. The terminal device according to claim 8, wherein the computer program is further configured to be executable by the processor to implement the following steps:
constructing each frame of ventricular model using each frame of CMR data, wherein the ventricular model comprises a plurality of vertices;
calculating a vertex correspondence between two adjacent frames of ventricular models using a registration algorithm; and
performing, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

10. The terminal device according to claim 8, wherein the computer program is further configured to be executable by the processor to implement the following steps:
constructing a filling ball model of the preset heart model;
establishing a mapping relationship between an element in the filling ball model and a surface vertex of the preset heart model;
determining a dynamic position of an element in the filling ball model according to the dynamic ventricular model;
determining a dynamic position of a surface vertex of the preset heart model according to the dynamic position of the element in the filling ball model; and
constructing a dynamic heart model of the patient to be operated according to the dynamic position of the surface vertex of the preset heart model.

11. The terminal device according to claim 10, wherein the computer program is further configured to be executable by the processor to implement the following steps:
triangulating the preset heart model to obtain a tetrahedral model;
providing a filling ballat a vertex of the tetrahedral model, and connecting filling balls through a three-dimensional spring; and
determining a model composed of the filling ball and the three-dimensional spring as the filling ball model.

12. The terminal device according to claim 8, wherein the computer program is further configured to be executable by the processor to implement the following steps:
performing coronary artery segmentation for the CCTA data using a level set algorithm to obtain a first coronary artery model;
extracting a center line in the first coronary artery model using a distance transform based three-dimensional center line extraction algorithm;
obtaining radius information of a cross section at each preset position of the center line; and
performing lofting processing on the center line according to the radius information of the cross section to obtain a second coronary artery model.

13. The terminal device according to claim 12, wherein the computer program is further configured to be executable by the processor to implement the following steps:
determining a cardiac portal for the coronary artery to enter in the dynamic heart model; and
registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.

14. The terminal device according to claim 13, wherein the computer program is further configured to be executable by the processor to implement the following step:
correcting the dynamic cardiovascular system model of the patient to be operated using a shape matching algorithm.

15. A computer readable storage medium having a computer program stored thereon, wherein the computer program is executed by a processor to implement the following steps:
obtaining cardiovascular magnetic resonance (CMR) data and coronary computed tomography angiography(CCTA) data of a patient to be operated;
constructing a dynamic ventricular model of the patient to be operated using the CMR data;
constructing a dynamic heart model of the patient to be operated according to the dynamic ventricular model and a preset heart model;
constructing a coronary artery model of the patient to be operated using the CCTA data; and
constructing a dynamic cardiovascular system model of the patient to be operated according to the dynamic heart model and the coronary artery model.

16. The computer readable storage medium according to claim 15, wherein the computer program is further executed by a processor to implement the following steps:
constructing each frame of ventricular model using each frame of CMR data, wherein the ventricular model comprises a plurality of vertices;
calculating a vertex correspondence between two adjacent frames of ventricular models using a registration algorithm; and
performing, according to the vertex correspondence between the two adjacent frames of ventricular models, an interpolation between the two adjacent frames of ventricular models using a linear interpolation algorithm to convert discrete respective frames of ventricular models into a continuous dynamic ventricular model.

17. The computer readable storage medium according to claim 15, wherein the computer program is further executed by a processor to implement the following steps:
constructing a filling ball model of the preset heart model;
establishing a mapping relationship between an element in the filling ball model and a surface vertex of the preset heart model;
determining a dynamic position of an element in the filling ball model according to the dynamic ventricular model;
determining a dynamic position of a surface vertex of the preset heart model according to the dynamic position of the element in the filling ball model; and
constructing a dynamic heart model of the patient to be operated according to the dynamic position of the surface vertex of the preset heart model.

18. The computer readable storage medium according to claim 17, wherein the computer program is further executed by a processor to implement the following steps:
triangulating the preset heart model to obtain a tetrahedral model;
providing a filling ballat a vertex of the tetrahedral model, and connecting filling balls through a three-dimensional spring; and
determining a model composed of the filling ball and the three-dimensional spring as the filling ball model.

19. The computer readable storage medium according to claim 15, wherein the computer program is further executed by a processor to implement the following steps:
performing coronary artery segmentation for the CCTA data using a level set algorithm to obtain a first coronary artery model;
extracting a center line in the first coronary artery model using a distance transform based three-dimensional center line extraction algorithm;
obtaining radius information of a cross section at each preset position of the center line; and
performing lofting processing on the center line according to the radius information of the cross section to obtain a second coronary artery model.

20. The computer readable storage medium according to claim 19, wherein the computer program is further executed by a processor to implement the following steps:
determining a cardiac portal for the coronary artery to enter in the dynamic heart model; and
registering the second coronary artery model to the dynamic heart model along the cardiac portal using a local constrained iterative nearest point algorithm, such that left and right vessel branches in the second coronary artery model correspond to corresponding ventricles of the dynamic heart model, so as to obtain a dynamic cardiovascular system model of the patient to be operated.
